SEQUENCE LISTING (9 sequences)

SEQ ID NO: 1 - DNA, 1122 bp, Arabidopsis thaliana
atgatatcacagagagaagaaagagaagagaagaagcagagagtgatgggagataagaaattgatttcatcttcttcttcttcctcggtttacgatactcgtatcaatcatcatcttcatcatcctccgtcttcttccgacgaaatctctcagtttctccggcatattttcgaccgttcttctcctttaccttcttactactccccggcgacgactacaacgacggcgtctttgattggtgtgcacgggagcggtgacccacatgcagataactcgagaagtctcgtttctcatcatccaccgtcagattctgtgcttatgtcgaaacgtgtcggagatttctctgaggttttaatcggcggaggatcaggctcagccgccgcgtgttttggtttctccggtggtggtaataataacaacgttcaaggaaatagctctgggactcgagtatcgtcttcttccgttggagctagtggcaacgagacagatgagtatgactgtgaaagcgaggaaggaggagaagctgtagttgatgaagctccctcttccaagtcaggtccttcttctcgtagttcatctaaaagatgcagagctgctgaagttcataatctctctgagaagaggaggagaagtagaattaatgaaaaaatgaaagctttacaaagtctcatccctaattcaaataagacggataaggcttcaatgcttgatgaagccattgagtatctgaaacagcttcagctccaagttcagatgttgactatgagaaatggaataaacttgcatcctttgtgtttacctggaactacattacacccattgcaactctctcagattcgaccccctgaagcaaccaatgatcctctgcttaatcataccaatcagtttgcttcgacttctaatgcaccggaaatgatcaatactgtggcttcttcatacgctttggaaccttctattcgcagtcactttggacctttccctctccttacttcacccgtggagatgagtcgggaaggtgggttaactcatccaaggttgaacattggtcattccaacgcaaacataaccggggaacaagctctgtttgatggacaacctgacctaaaagatcgaattacttga

SEQ ID NO: 2 - DNA, 22 bp, Arabidopsis thaliana
ccttacttcacccgtggagatg

SEQ ID NO: 3 - DNA, 21 bp, Arabidopsis thaliana
gcgttggaatgaccaatgttc

SEQ ID NO: 4 - DNA, 23 bp, Arabidopsis thaliana
aagtggacccctaaagacgatct

SEQ ID NO: 5 - DNA, 23 bp, Arabidopsis thaliana
gtcgatgagagggatgttttcac

SEQ ID NO: 6 - DNA, 22 bp, Arabidopsis thaliana
tgagttcctcaccggaagtctt

SEQ ID NO: 7 - DNA, 17 bp, Arabidopsis thaliana
cgagccgccttgagctt

SEQ ID NO: 8 - DNA, 43 bp, Arabidopsis thaliana
gcgacgcgtaattactactaccatgatatcacagagagaagaa

SEQ ID NO: 9 - DNA, 29 bp, Arabidopsis thaliana
gcggggcccagtaattcgatcttttaggt

(19) United States
(12) Patent Application Publication
(10) Pub. No.: US 2009/0019607 A1
(43) Pub. Date: Jan. 15, 2009

(54) TRANSGENIC PLANT CELLS EXPRESSING A TRANSCRIPTION FACTOR

(75) Inventors: Ian Alexander Graham, York (GB); Steven Penfield, York (GB)

Correspondence Address: SPECKMAN LAW GROUP PLLC, 1201 THIRD AVENUE, SUITE 330, SEATTLE, WA, 98101, US

(73) Assignee: THE UNIVERSITY OF YORK, York (GB)

(21) Appl. No.: 12/091,446

(22) PCT Filed: Oct. 25, 2006
(86) PCT No.: PCT/GB06/03971

§ 371 Date: Sep. 8, 2008

(30) Foreign Application Priority Data
Oct. 25, 2005 (GB) 0521691.6
Nov. 19, 2005 (GB) 0523593.2

(51) Int. Cl. A01H 5/00; C12N 5/04; C12Q 1/68

(52) U.S. Cl. 800/298; 435/419; 435/6

(57) ABSTRACT

We describe a transgenic plant with altered expression of a gene that encodes a sequence variant of a transcription factor and which has altered seed dormancy.

The invention relates to a transgenic plant with altered expression of a gene that encodes a sequence variant of a transcription factor and the use of the transcription factor in quantitative trait analysis (QTL).

A key feature of plant adaptive fitness is the ability to synchronise the onset of vegetative and reproductive development with seasonal changes in the environment. The commencement of vegetative development is controlled by a period of quiescence in the mature seed known as seed dormancy. During dormancy, seed germination does not occur even though local conditions are capable of supporting radicle emergence from the seed coat. The period of dormancy of many plant seeds is terminated by environmental signals including light, temperature and nutrient availability, a system adapted to the promotion of germination only when conditions are optimal for seedling establishment and reproductive success. In particular the role of light and temperature in the promotion of germination in dormant seeds is highly conserved among seed plants from angiosperms to gymnosperms, demonstrating the importance of germination control as a vital adaptive trait in plants [1].

One of the primary objectives of the commercial grower is to regulate the growth and development of plants to maximize the value of a crop. Growers need to control the rate (timing) of development/germination, flowering, plant stature (height) and architecture (branching) and this can be achieved by altering cellular, biochemical and molecular mechanisms of growth regulation. A problem associated with certain plant species is sprouting/precocious germination and one solution to this problem is to control seed dormancy.

US 2002/0148008 disclose the development of genetically modified wheat seed, in which the expression levels of VP1 are modulated to regulate seed dormancy. VP1 is a transcriptionally regulated gene essential for formation of seed dormancy; it is a transcription factor which acts in the abscisic acid signalling system. Site-directed mutagenesis of the gene results in a protein which comprises an amino acid sequence having deletions, substitutions or additions. By introducing mutants of this gene into varieties of wheat, the expression of its protein can be altered, consequently the degree of abscisic acid-sensitivity of the seeds in those varieties can be altered and thus the degree of dormancy of the seed.

WO02/077163 describes the over expression of the gene ABI5 in plants such as Arabidopsis thaliana, to prevent precocious seed germination. ABI5 encodes a putative transcription factor of the basic leucine zipper (bZIP) family. The bZIP region of ABI5 shows extensive homology to previously characterised plant (bZIP) transcription factors capable of activating reporter genes containing ABA-responsive DNA elements (ABREs). ABI5 has been shown to confer an enhanced response to exogenous abscisic acid during germination. As a key component in ABA-triggered processes, ABI5 protein accumulation, phosphorylation, stability and activity are highly regulated by ABA during germination and early seedling growth. Plants which over express ABI5 are hypersensitive to abscisic acid and therefore respond to very low levels of this phytohormone, some three times lower, which would have no effect on wild type plants.

Moreover, it is desirable to develop plant varieties that include alleles of genes that confer beneficial agronomic traits on the plant variety, for example plant varieties that do not have the phenotype of sprouting/precocious germination. Many phenotypic traits of agronomic value are controlled by single genes and therefore knowledge of a specific genotype allows the prediction of a specific phenotype associated with that genotype. These phenotypes are referred to as discontinuous phenotypes. Other traits do not fall into this category because they are controlled by multiple genes. These traits are referred to as continuous traits and cannot be analysed in the same predictable way. They are often referred to as quantitative traits. The genetic loci controlling these traits are called quantitative trait loci or simply QTL. Many important traits such as crop yield are controlled by QTL's. QTL's are therefore genetic markers that are strongly associated with a highly desirable agronomic trait. The most valuable QTL marker is one that detects a specific gene, or variant, which can be readily detected. In many cases QTL's are not associated with a specific gene but rather genetic loci that are near to a gene, for example a microsatellite sequence. However, as noted above, quantitative traits can be controlled by several genes the combined expression of which results in the desirable phenotypic trait. Nevertheless, the identification of gene markers that contribute to the qualitative trait is desirable.

We disclose a further transcription factor involved in regulating seed germination. The basic helix-loop-helix transcription factor called SPATULA (SPT) is involved in the control of the germination of dormant seeds by light and temperature. We show that SPT is a multifunctional transcription factor, acting as a light stable repressor of GA3ox expression controlling seed responses to cold stratification, and to a lesser extent red light. SPT is the first described regulator of cold stratification in plants. We also describe a mutational variant of SPT called spt-2 that has reduced germination and is not responsive to cold stratification. The spt-2 mutation has a semi-dominant effect on seed germination and therefore the SPT-2 protein is likely to be of use in the modification of dormancy characteristics of various domesticated plant species. Furthermore, the SPT and variants thereof represent a new QTL associated withsprouting/precocious germination in agronomically important plant species.

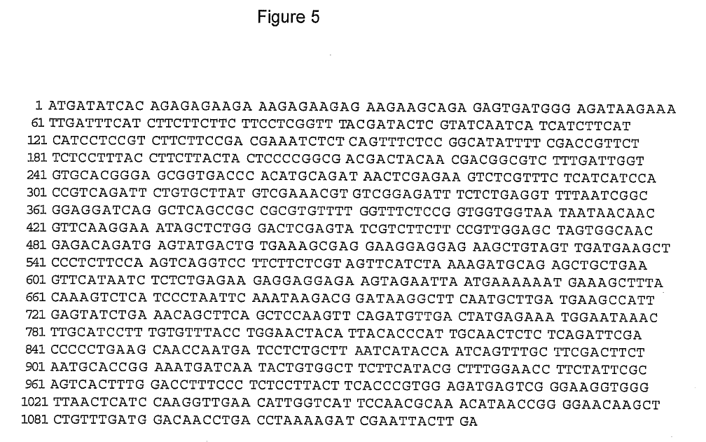

According to an aspect of the invention there is provided a plant comprising a genetically modified cell wherein the genome of said cell is modified by the inclusion of a nucleic acid molecule comprising a nucleic acid sequence represented in FIG. 5, wherein said nucleic acid molecule encodes a transcription factor polypeptide that is a variant polypeptide from that encoded by the nucleic acid sequence in FIG. 5, which variant polypeptide comprises an amino acid deletion or substitution of amino acid residue 209.

According to a further aspect of the invention there is provided a plant comprising a genetically modified cell wherein the genome of said cell is modified by the inclusion of a nucleic acid molecule that hybridises under stringent hybridisation conditions to the sequence in FIG. 5 and which includes a deletion or substitution of amino acid residue 209, or an equivalent amino acid residue in a homologous nucleic acid molecule.

Hybridization of a nucleic acid molecule occurs when two complementary nucleic acid molecules undergo an amount of hydrogen bonding to each other. The stringency of hybridization can vary according to the environmental conditions surrounding the nucleic acids, the nature of the hybridization method, and the composition and length of the nucleic acid molecules used. Calculations regarding hybridization conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology—Hybridization with Nucleic Acid Probes Part I, Chapter 2 (Elsevier, New York, 1993). The Tₘ is the temperature at which 50% of a given strand of a nucleic acid molecule is hybridized to its complementary strand. The following is an exemplary set of hybridization conditions and is not limiting:

Very High Stringency
(allows sequences that share at least 90% identity to hybridize)

| | |
|---|---|
| Hybridization: | 5x SSC at 65° C. for 16 hours |
| Wash twice: | 2x SSC at room temperature (RT) for 15 minutes each |
| Wash twice: | 0.5x SSC at 65° C. for 20 minutes each |

High Stringency
(allows sequences that share at least 80% identity to hybridize)

| | |
|---|---|
| Hybridization: | 5x-6x SSC at 65° C.-70° C. for 16-20 hours |
| Wash twice: | 2x SSC at RT for 5-20 minutes each |
| Wash twice: | 1x SSC at 55° C.-70° C. for 30 minutes each |

Low Stringency
(allows sequences that share at least 50% identity to hybridize)

| | |
|---|---|
| Hybridization: | 6x SSC at RT to 55° C. for 16-20 hours |
| Wash at least twice: | 2x-3x SSC at RT to 55° C. for 20-30 minutes each. |

In a preferred embodiment of the invention said substitution is the replacement of amino acid residue 209 with a basic amino acid residue.

In a further preferred embodiment of the invention said amino acid residue 209 is arginine. Preferably, said arginine amino acid residue is replaced with a basic amino acid residue that is not arginine. Preferably, said amino acid residue is lysine or histidine.

In a preferred embodiment of the invention said plant comprises a nucleic acid molecule that encodes a transcription factor the activity of which is modulated.

In a preferred embodiment of the invention said transcription factor activity is increased when compared to a non-transgenic reference plant of the same species. Preferably said activity is increased by at least about 2-fold above a basal level of activity. More preferably said activity is increased by at least about 5 fold; 10 fold; 20 fold, 30 fold, 40 fold, 50 fold. Preferably said activity is increased by between at least 50 fold and 100 fold. Preferably said increase is greater than 100-fold.

It will be apparent that means to increase the activity of a polypeptide encoded by a nucleic acid molecule are known to the skilled artisan. For example, and not by limitation, increasing the gene dosage by providing a cell with multiple copies of said gene. Alternatively or in addition, a gene(s) may be placed under the control of a powerful promoter sequence or an inducible promoter sequence to elevate expression of mRNA encoded by said gene. The modulation of mRNA stability is also a mechanism used to alter the steady state levels of an mRNA molecule, typically via alteration to the 5′ or 3′ untranslated regions of the mRNA.

In a preferred embodiment of the invention said nucleic acid molecule is a vector adapted for transformation of said plant cell. Preferably said vector is adapted for the over expression of said nucleic acid molecule encoding said transcription factor.

Suitable vectors can be constructed, containing appropriate regulatory sequences, including promoter sequences, terminator fragments, polyadenylation sequences, enhancer sequences, marker genes and other sequences as appropriate. For further details see, for example, Molecular Cloning: Laboratory Manual: 2^{nd} edition, Sambrook et al. 1989, Cold Spring Habor Laboratory Press or Current Protocols in Molecular Biology, Second Edition, Ausubel et al. Eds., John Wiley & Sons, 1992.

Specifically included are shuttle vectors by which is meant a DNA vehicle capable, naturally or by design, of replication in two different host organisms, which may be selected from actinomycetes and related species, bacteria and eukaryotic (e.g. higher plant, mammalian, yeast or fungal cells).

Preferably the nucleic acid in the vector is under the control of, and operably linked to, an appropriate promoter or other regulatory elements for transcription in a host cell such as a microbial, (e.g. bacterial), or plant cell. The vector may be a bi-functional expression vector which functions in multiple hosts. In the case of GTase genomic DNA this may contain its own promoter or other regulatory elements and in the case of cDNA this may be under the control of an appropriate promoter or other regulatory elements for expression in the host cell.

By “promoter” is meant a nucleotide sequence upstream from the transcriptional initiation site and which contains all the regulatory regions required for transcription. Suitable promoters include constitutive, tissue-specific, inducible, developmental or other promoters for expression in plant cells comprised in plants depending on design. Such promoters include viral, fungal, bacterial, animal and plant-derived promoters capable of functioning in plant cells.

Constitutive promoters include, for example CaMV 35S promoter (Odell et al. (1985) Nature 313, 9810-812); rice actin (McElroy et al. (1990) Plant Cell 2: 163-171); ubiquitin (Christian et al. (1989) Plant Mol. Biol. 18 (675-689); pEMU (Last et al. (1991) Theor Appl. Genet. 81: 581-588); MAS (Velten et al. (1984) EMBO J. 3. 2723-2730); ALS promoter (U.S. application Ser. No. 08/409,297), and the like. Other constitutive promoters include those in U.S. Pat. Nos. 5,608,149; 5,608,144; 5,604,121; 5,569,597; 5,466,785; 5,399,680, 5,268,463; and 5,608,142.

Chemical-regulated promoters can be used to modulate the expression of a gene in a plant through the application of an exogenous chemical regulator. Depending upon the objective, the promoter may be a chemical-inducible promoter, where application of the chemical induced gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88: 10421-10425 and McNellis et al. (1998) Plant J. 14(2): 247-257) and tetracycline-inducible and tetracycline-repressible promoters (see, for example, Gatz et al. (1991) Mol. Gen. Genet. 227: 229-237, and U.S. Pat. Nos. 5,814,618 and 5,789,156, herein incorporated by reference.

Where enhanced expression in particular tissues is desired, tissue-specific promoters can be utilised. Tissue-specific promoters include those described by Yamamoto et al. (1997) Plant J. 12(2): 255-265; Kawamata et al. (1997) Plant Cell Physiol. 38(7): 792-803; Hansen et al. (1997) Mol. Gen. Genet. 254(3): 337-343; Russell et al. (1997) Transgenic Res. 6(2): 157-168; Rinehart et al. (1996) Plant Physiol. 112(3): 1331-1341; Van Camp et al. (1996) Plant Physiol. 112(2): 525-535; Canevascni et al. (1996) Plant Physiol. 112(2): 513-524; Yamamoto et al. (1994) Plant Cell Physiol. 35(5): 773-778; Lam (1994) Results Probl. Cell Differ. 20: 181-196; Orozco et al. (1993) Plant Mol. Biol. 23(6): 1129-1138; Mutsuoka et al. (1993) Proc. Natl. Acad. Sci. USA 90 (20): 9586-9590; and Guevara-Garcia et al (1993) Plant J. 4(3): 495-50.

“Operably linked” means joined as part of the same nucleic acid molecule, suitably positioned and oriented for transcription to be initiated from the promoter. DNA operably linked to a promoter is “under transcriptional initiation regulation” of the promoter. In a preferred aspect, the promoter is an inducible promoter or a developmentally regulated promoter.

In a preferred embodiment of the invention said nucleic acid molecule is controlled by a seed specific promoter.

Particular of interest in the present context are nucleic acid constructs which operate as plant vectors. Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148. Suitable vectors may include plant viral-derived vectors (see e.g. EP-A-194809).

If desired, selectable genetic markers may be included in the construct, such as those that confer selectable phenotypes such as resistance to antibodies or herbicides (e.g. kanamycin, hygromycin, phosphinotricin, chlorsulfuron, methotrexate, gentamycin, spectinomycin, imidazolinones and glyphosate).

Plants transformed with a DNA construct of the invention may be produced by standard techniques known in the art for the genetic manipulation of plants. DNA can be introduced into plant cells using any suitable technology, such as a disarmed Ti-plasmid vector carried by Agrobacterium exploiting its natural gene transferability (EP-A-270355, EP-A-0116718, NAR 12(22):8711-87215 (1984), Townsend et al., U.S. Pat. No. 5,563,055); particle or microprojectile bombardment (U.S. Pat. No. 5,100,792, EP-A-444882, EP-A-434616; Sanford et al, U.S. Pat. No. 4,945,050; Tomes et al. (1995) “Direct DNA Transfer into Intact Plant Cells via Microprojectile Bombardment”, in Plant Cell, Tissue and Organ Culture: Fundamental Methods, ed. Gamborg and Phillips (Springer-Verlag, Berlin); and McCabe et al. (1988) Biotechnology 6: 923-926); microinjection (WO 92/09696, WO 94/00583, EP 331083, EP 175966, Green et al. 91987) Plant Tissue and Cell Culture, Academic Press, Crossway et al. (1986) Biotechniques 4:320-334); electroporation (EP 290395, WO 8706614, Riggs et al. (1986) Proc. Natl. Acad. Sci. USA 83:5602-5606; D'Halluin et al. 91992). Plant Cell 4:1495-1505) other forms of direct DNA uptake (DE 4005152, WO 9012096, U.S. Pat. No. 4,684,611, Paszkowski et al. (1984) EMBO J. 3:2717-2722); liposome-mediated DNA uptake (e.g. Freeman et al (1984) Plant Cell Physiol, 29:1353); or the vortexing method (e.g. Kindle (1990) Proc. Nat. Acad. Sci. USA 87:1228). Physical methods for the transformation of plant cells are reviewed in Oard (1991) Biotech. Adv. 9:1-11. See generally, Weissinger et al. (1988) Ann. Rev. Genet. 22:421-477; Sanford et al. (1987) Particulate Sciences and Technology 5:27-37; Christou et al. (1988) Plant Physiol. 87:671-674; McCabe et al. (1988) Bio/Technology 6:923-926; Finer and McMullen (1991) In Vitro Cell Dev. Biol. 27P:175-182; Singh et al. (1988) Theor. Appl. Genet. 96:319-324; Datta et al. (1990) Biotechnology 8:736-740; Klein et al. (1988) Proc. Natl. Acad. Sci. USA 85: 4305-4309; Klein et al. (1988) Biotechnology 6:559-563; Tomes, U.S. Pat. No. 5,240,855; Buising et al. U.S. Pat. Nos. 5,322, 783 and 5,324,646; Klein et al. (1988) Plant Physiol 91: 440-444; Fromm et al (1990) Biotechnology 8:833-839; Hooykaas-Von Slogteren et al. 91984). Nature (London) 311:763-764; Bytebier et al. (1987) Proc. Natl. Acad. Sci. USA 84:5345-5349; De Wet et al. (1985) in The Experimental Manipulation of Ovule Tissues ed. Chapman et al. (Longman, N.Y.), pp. 197-209; Kaeppler et al. (1990) Plant Cell Reports 9:415-418 and Kaeppler et al. (1992) Theor. Appl. Genet. 84:560-566; Li et al. (1993) Plant Cell Reports 12: 250-255 and Christou and Ford (1995) Annals of Botany 75: 407-413; Osjoda et al. (1996) Nature Biotechnology 14:745-750, all of which are herein incorporated by reference.

Agrobacterium transformation is widely used by those skilled in the art to transform dicotyledonous species. Recently, there has been substantial progress towards the routine production of stable, fertile transgenic plants in almost all economically relevant monocot plants (Toriyama et al. (1988) Bio/Technology 6: 1072-1074; Zhang et al. (1988) Plant Cell rep. 7379-384; Zhang et al. (1988) Theor. Appl. Genet. 76:835-840; Shimamoto et al. (1989) Nature 338:274-276; Datta et al. (1990) Bio/Technology 8: 736-740; Christou et al. (1991) Bio/Technology 9:957-962; Peng et al (1991) International Rice Research Institute, Manila, Philippines, pp. 563-574; Cao et al. (1992) Plant Cell Rep. 11: 585-591; Li et al. (1993) Plant Cell Rep. 12: 250-255; Rathore et al. (1993) Plant Mol. Biol. 21:871-884; Fromm et al (1990) Bio/Technology 8:833-839; Gordon Kamm et al. (1990) Plant Cell 2:603-618; D'Halluin et al. (1992) Plant Cell 4:1495-1505; Walters et al. (1992) Plant Mol. Biol. 18:189-200; Koziel et al. (1993). Biotechnology 11194-200; Vasil, I. K. (1994) Plant Mol. Biol. 25:925-937; Weeks et al (1993) Plant Physiol. 102:1077-1084; Somers et al. (1992) Bio/Technology 10:1589-1594; WO 92/14828. In particular, Agrobacterium mediated transformation is now emerging also as a highly efficient transformation method in monocots. (Hiei, et al. (1994) The Plant Journal 6:271-282). See also, Shimamoto, K. (1994) Current Opinion in Biotechnology 5:158-162; Vasil, et al. (1992) Bio/Technology 10:667-674; Vain, et al. (1995) Biotechnology Advances 13(4):653-671; Vasil, et al. (1996) Nature Biotechnology 14: 702).

Microprojectile bombardment, electroporation and direct DNA uptake are preferred where Agrobacterium is inefficient or ineffective. Alternatively, a combination of different techniques may be employed to enhance the efficiency of the transformation process, e.g. bombardment with Agrobacterium-coated microparticles (EP-A-486234) or microprojectile bombardment to induce wounding followed by co-cultivation with Agrobacterium (EP-A-486233).

In a preferred embodiment of the invention said plant is selected from the group consisting of: corn (Zea mays), canola (Brassica napus, Brassica rapa ssp.), flax (Linum usitatissimum), alfalfa (Medicago sativa), rice (Oryza saliva), rye (Secale cerale), sorghum (Sorghum bicolor, Sorghum vulgare), sunflower (Helianthus annus), wheat (Tritium aestivum), soybean (Glycine max), tobacco (Nicotiana tabacum), potato (Solanum tuberosum), peanuts (Arachis hypogaea), cotton (Gossypium hirsutum), sweet potato (Iopmoea batatus), cassava (Manihot esculenta), coffee (Cofea spp.), coconut (Cocos nucifera), pineapple (Anana comosus), citris tree (Citrus spp.) cocoa (Theobroma cacao), tea (Camellia senensis), banana (Musa spp.), avacado (Persea americana), fig (Ficus casica), guava (Psidium guajava), mango (Mangifer indica), olive (Olea europaea), papaya (Carica papaya), cashew (Anacardium occidentale), macadamia (Macadamia intergrifolia), almond (Prunus amygdalus), sugar beets (Beta vulgaris), oats, barley, vegetables.

Preferably, plants of the present invention are crop plants (for example, cereals and pulses, maize, wheat, potatoes, tapioca, rice, sorghum, millet, cassava, barley, pea), and other root, tuber or seed crops. Important seed crops are oil-seed rape, sugar beet, maize, sunflower, soybean, sorghum, and flax (linseed).

Horticultural plants to which the present invention may be applied may include lettuce, endive, and vegetable brassicas including cabbage, broccoli, and cauliflower. The present invention may be applied in tobacco, cucurbits, carrot, strawberry, sunflower, tomato, pepper. Also included are ornamental plants e.g Agastache, Ageratum, Althea rosea, Alyssum, Amaranthus, Antirrhinum, Asclepias, Asters, Balsam, Basil (ornamental), Begonia semperflorens, Begonia elatior, Begonia tuberous, Bidens, Calceolaria rugosa, Calendula, Callistephus, Canna, Capsicum, Carnation, Carthamus, Celosia, Centaurea, Chrysanthemum, Cineraria maritima, Cleome, Coleus, Coreopsis, Cosmos, Cosmos sulphureum, Cuphea, Cynoglossum, Dahlia, Dianthus barbatus, Dianthuscariophyllus, Dianthusplumarius, Dianthus sinensis, Delphinium, Diasca, Didiscus, Echium, Euphorbia, Exacum, Ficoides, Flower Kale, Fuchsia, Gazania, Geranium, Gerbera, Godetia, Grasses (ornamental), Helianthus, Heliotrope, Helichrysum, Impatiens, Impatiens New Guinea, Ipomea, Lagerstroemia, Larkspur, Lavender, Lavatera, Leucanthemum, Lilium, Linaria, Lisianthus, Lobelia, Lobelia speciosa, Marigold, African, Marigold, French, Matthiola, Mesambrianthemum, Mimulus, Molucella, Nasturtium, Nemesia, Nicotiana, Nierembergia, Oxypetalum, Papaver, ornamental, Pelargonium, Pentas Pepper, ornemental, Petunias, Petunia double, Petunia gigantiflora, Petunia grandiflora, Petunia milliflora, Petunia multiflora, Phlox, Pinks, Platycodon, Portulacca, Ricinus, Rudbeckia, Sanvitalia, Salvia, Salvia coccinea, Salvia farinacea, Salvia patens, Salvia splendens, Schizanthus, Snapdragon, Solanum, Statice, Stocks, Sweet Peas, Swiss Chard, T.E.P. (Tagetes Erecta×Patula), Tagetes erecta, Tagetes patula, Tagetes signata, Thlaspi, Tithonia, Tobacco, ornamental, Verbascum, Verbena, Vinca, Zinniapetunia.

Grain plants that provide seeds of interest include oil-seed plants and leguminous plants. Seeds of interest include grain seeds, such as corn, wheat, barley, rice, sorghum, rye, etc. Oil-seed plants include cotton, soybean, safflower, sunflower, Brassica, maize, alfalfa, palm, coconut, etc. Leguminous plants include beans and peas. Beans include guar, locust bean, fenugreek, soybean, garden beans, cowpea, mungbean, lima bean, fava been, lentils, chickpea, etc.

In a preferred embodiment of the invention said plant has reduced germination when compared to a non transgenic reference plant of the same species.

In a further preferred embodiment of the invention said plant has reduced response to cold stratification when compared to a non transgenic reference plant of the same species.

According to a yet further aspect of the invention there is provided a seed obtained from a plant according to the invention.

According to an aspect of the invention there is provided a plant cell wherein said cell is modified by the inclusion of a nucleic acid molecule comprising a nucleic acid sequence represented in FIG. 5A, wherein said nucleic acid molecule encodes a transcription factor polypeptide that is a variant polypeptide from that encoded by the nucleic acid sequence in FIG. 5A, which variant polypeptide comprises an amino acid deletion or substitution of amino acid residue 209.

According to a further aspect of the invention there is provided a plant cell wherein said cell is modified by the inclusion of a nucleic acid molecule that hybridises under stringent hybridisation conditions to the sequence in FIG. 5A and which includes a deletion or substitution of amino acid residue 209, or an equivalent amino acid residue in a homologous nucleic acid molecule.

According to a further aspect of the invention there is provided the use of a gene encoded by a nucleic acid molecule as represented by the nucleic acid sequence in FIG. 5a, or a nucleic acid molecule that hybridises to the sequence in FIG. 5a and encodes a polypeptide with transcription factor activity as a quantitative trait locus.

According to a yet further aspect of the invention there is provided a method for the identification of a genetic marker associated with sprouting/precocious germination wherein said locus is associated with a nucleic acid sequence selected from the group consisting of:
a) a nucleic acid molecule comprising a nucleic acid sequence as represented in FIG. 5a;
b) a nucleic acid molecule that hybridises to the nucleic acid molecule in a) under stringent hybridisation conditions and that encodes a polypeptide with transcription factor activity;
c) a nucleic acid molecule comprising a nucleic acid sequence that is degenerate as a result of the genetic code to the sequences as defined in (a) and (b) above; comprising the steps of:
   i) providing a sample comprising a plant cell wherein said plant cell is derived from a plant that does not express a sprouting/precocious germination phenotype;
   ii) comparing the sequence of the nucleic acid molecule in said sample to a nucleic acid sequence of a nucleic acid molecule of a plant that does express a sprouting/precocious germination phenotype.

According to a further aspect of the invention there is provided a method to produce a plant variety that does not express a sprouting/precocious germination phenotype comprising the steps of:
i) mutagenesis of wild-type seed from a plant that does express a sprouting/precocious germination phenotype;
ii) cultivation of the seed in i) to produce a first and subsequent generations of plants;
iii) obtaining seed from the first generation plant;
iv) determining if the seed from said first and subsequent generations of plants do not express a sprouting/precocious germination phenotype;
v) obtaining a sample and analysing the nucleic acid sequence of a nucleic acid molecule selected from the group consisting of:
   a) a nucleic acid molecule comprising a nucleic acid sequence as represented in FIG. 5a;
   b) a nucleic acid molecule that hybridises to the nucleic acid molecule in i) under stringent hybridisation conditions and that encodes a polypeptide with transcription factor activity;
   c) a nucleic acid molecule comprising a nucleic acid sequence that is degenerate as a result of the genetic code to the sequences as defined in (i) and (ii) above;
vi) comparing the sequence of the nucleic acid molecule in said sample to a nucleic acid sequence of a nucleic acid molecule of a plant that does express a sprouting/precocious germination phenotype.

In a preferred method of the invention said nucleic acid molecule is analysed by a method comprising the steps of:
i) extracting nucleic acid from said mutated plants;
ii) amplification of a part of said nucleic acid molecule by a polymerase chain reaction;
iii) forming a preparation comprising the amplified nucleic acid and nucleic acid extracted from wild-type seed to form heteroduplex nucleic acid;
iv) incubating said preparation with a single stranded nuclease that cuts at a region of heteroduplex nucleic acid to identify the mismatch in said heteroduplex; and
v) determining the site of the mismatch in said nucleic acid heteroduplex.

In a preferred method of the invention said nucleic acid molecule comprises a nucleic acid sequence as represented in FIG. 5a. Preferably said nucleic acid molecule consists of the nucleic acid sequence in FIG. 5a.

In a preferred method of the invention said plant cell or seed is from wheat, barley or oil seed rape.

An embodiment of the invention will now be described by example only and with reference to the following figures:

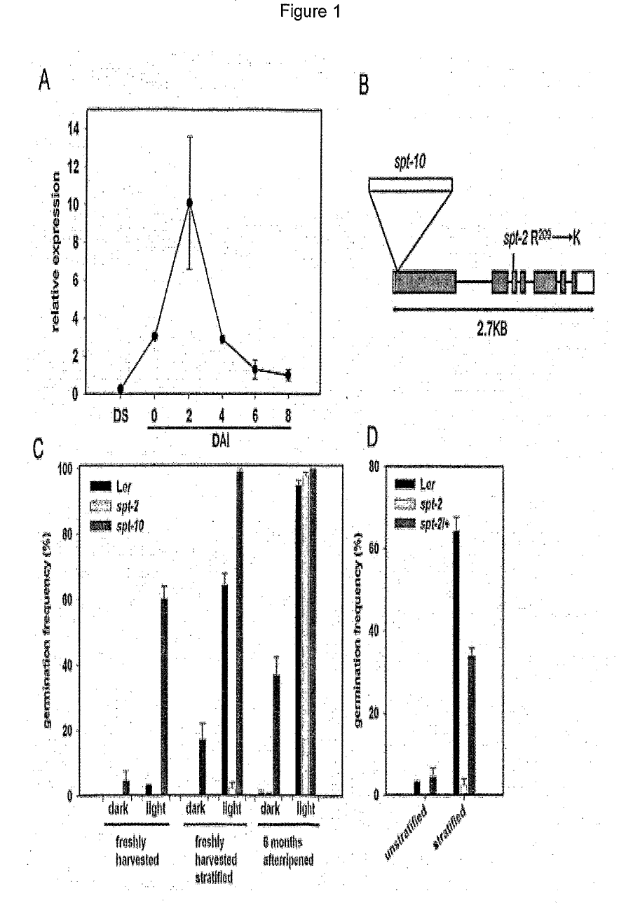

FIG. 1: SPATULA is expressed in imbibed seed and controls the response to stratification. A. Real-time RT-PCR showing SPT expression during seed imbibition and germination. DS—dry seed, DAI—days after imbibition. B. Scheme to illustrate the position of the spt-2 and spt-10 mutants. C. The germination phenotype of freshly harvested and afterripened wild type, spt-2 and spt-10 seeds in response to cold stratification and white light. D. The response of freshly harvested seeds heterozygous for the spt-2 mutation to stratification in white light;

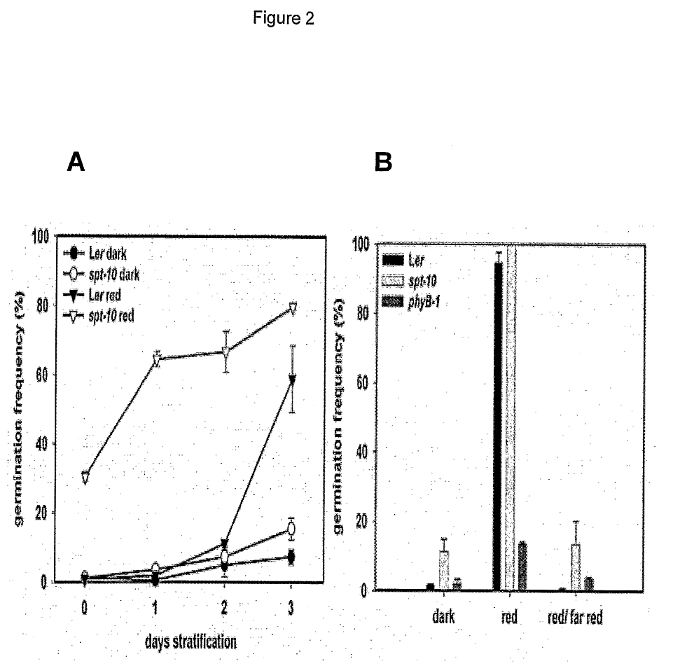

FIG. 2: A. The response of freshly harvested Ler and spt-10 seed to stratification in the dark and after red light treatment. B. The germination of stratified Ler, spt-10 and phyB-1 seed in the dark, after a red light pulse, and after a red light pulse followed by a far red pulse;

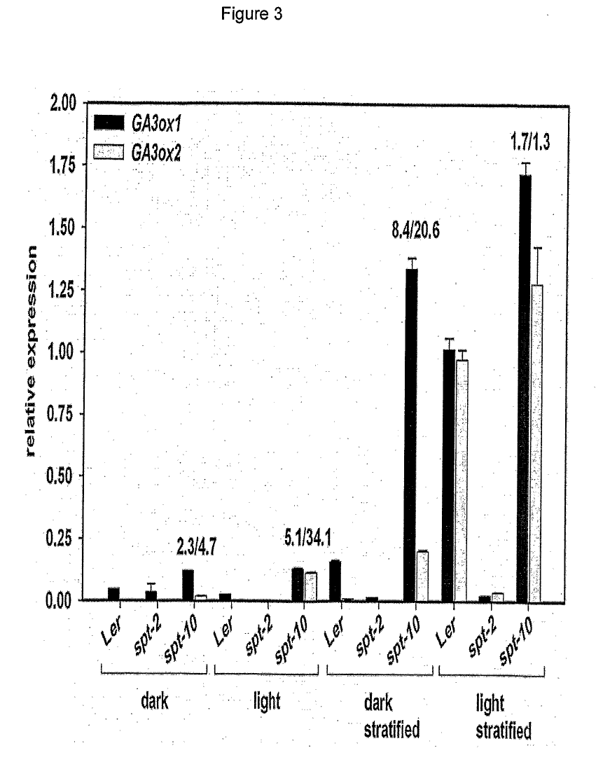

FIG. 3: Real-time RT-PCR to show the transcript abundance of GA3ox1 and GA3ox2 in imbibed seeds prior to germination. Figures above the spt-10 data show the relative increase in the spt-10 mutant in GA3ox1 and GA3ox2 expression respectively compared to wild type under the same conditions. Expression is shown relative to the expression in light treated stratified wild type seed which was set to 1;

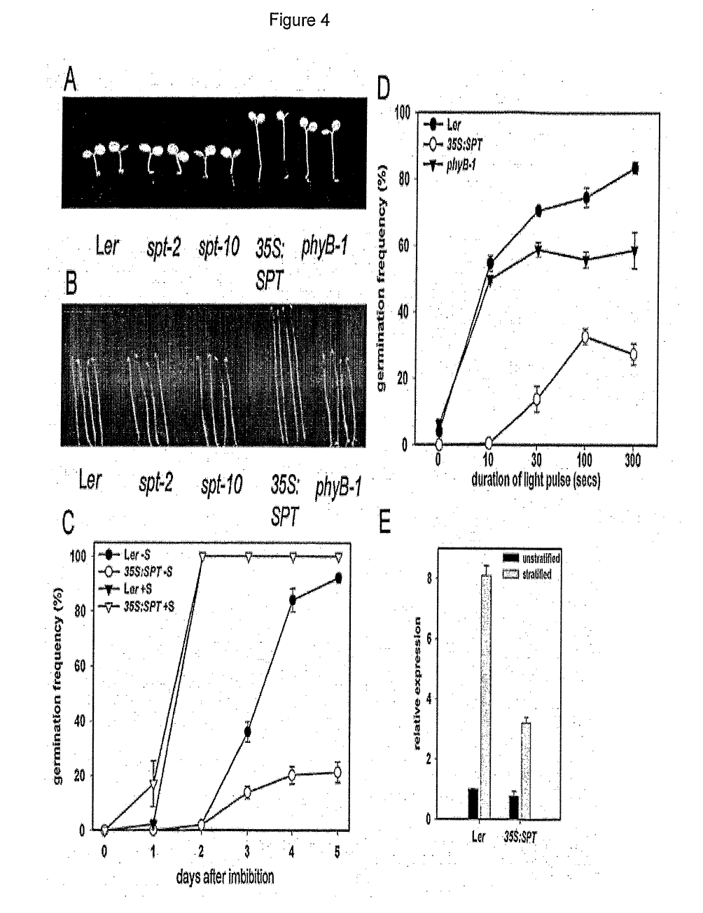

FIG. 4: The overexpression of SPT. A. The seedling morphology of spt mutants and overexpressors after 5 days growth in white light. B. Dark grown seedling morphology of wild type, 35S:SPT and the spt mutants after 5 days. C. The germination of 1 week afterripened Ler and 35S:SPT seed in white light. −S—without 3 nights stratification, +S—with 3 nights stratification. D. The germination of freshly harvested stratified wild type, phyB-1 and 35S:SPT seed in the dark, and after red light pulses of increasing duration. E. The expression of GA3ox in imbibed seeds of wild type and 35S:SPT after 24 h in white light at 20° C., with and without 3 nights prior stratification;

FIG. 5 is the nucleic acid sequence of a cDNA that encodes wild-type SPT.

### MATERIALS AND METHODS

Plant Material. spt-2 and pil5-1 and phyB-1 seeds were obtained from the Nottingham Arabidopsis Stock Centre (NASC) and have been previously described (Alvarez and Smyth, 1998; Oh et al., 2004; Reed et al., 1993). The spt-10 insertion line corresponds to line ET7451 from the Cold Spring harbour enhancer-trap collection (genetrap.cshl.org). The presence of the insertion was followed by the spatula carpel phenotype and the absence of the wild type SPT transcript. pil5-2 corresponds to line SALK_{—}131872. This line segregates kanamycin resistance 3:1 and exhibits similar seed and seedling phenotypes to pil5-1 (data not shown).

Germination assays. Seed for germination assays was harvested from plants grown simultaneously in glasshouse conditions with supplementary lighting to ensure a 16 hour photoperiod. The term freshly harvested refers to seed collected from siliques that had just changed from green to brown. These were sown within 48 hours of harvest for germination assays. Both Ler and Co10 were found to be dormant at this time. Seed was sown 0.9% (wt/vol) water-agar medium and stratified where indicated in the dark at 4-6° C. Wrapping plates immediately in three layers of foil after sowing but before imbibition was found to be essential to retain the light requirement for germination in wild type (data not shown). Germination was scored by radicle emergence after 5 days on 5 batches of 40-100 seed from each genotype, each batch being obtained from one individual plant. Growth conditions under white light were 20° C. 16 h photoperiod at a photon fluence rate of 75 μmol m⁻² s⁻¹. For experiments with red light, seeds imbibed in the dark were warmed to ambient temperature exposed to a pulse of continuous monochromatic red LEDs (PEAK 660 nm, 40 μmol m⁻² s⁻¹) as indicated, before re-wrapping and incubating in the dark at 20° C. for 5 days. Seed afterripening took place in dark storage in the laboratory, typically at 1 8-20° C. All data points represent the mean and standard error of 5 seed batches (3 seed batches for the red/far red reversibility experiment). Experiments were repeated several times and similar results obtained.

Seedling growth assays For all experiments with seedlings 20-30 seeds were sown on Gilroy-phytagel or water-agar plates. In the fluence response assays germination was stimulated by a pulse of white light following a 4 day period of stratification at 4° C. Plates were then kept in darkness or transferred to the appropriate light treatment after a 24 hour period. Hypocotyl and cotyledon measurements were performed on seedlings 7 days post-imbibition using ImageJ to the nearest 0.5 mm. For de-etiolation experiments seedlings kept at 20° C. were exposed to continuous monochromatic red LEDs (PEAK 660 nm, 0-100 μmol m⁻² s⁻¹), far-red LEDs (PEAK 756 nm, 0-120 μmol m⁻² s⁻¹), blue LEDs (PEAK 439 and 455 nm, 0-120 μmol m⁻² s⁻¹), or white light provided by fluorescent tubes (Sylviana; PEAK 434/455/631/707 nm, 0-100 μmol m⁻² s⁻¹).

Construction of SPT Overexpressing Plants. The SPT cDNA was obtained as a pBLUESCRIPT clone from a cDNA library constructed from 2 day old germinating seeds (I. A. Graham, unpublished) and was confirmed as full length by sequencing with standard primers. The sequence is 100% identical to that described in genbank entry AF319540. Using standard molecular biology techniques the SPT cDNA was excised as a BamHI EcoRI fragment and cloned into the pGREENII-0029 35S vector, containing a double cauliflower mosaic virus (CaMV) 35S promoter [30]. This was transformed into Agrobacterium strain GV3101 and into Arabidopsis Landsberg erecta by the floral dip method. 20 independent transgenic lines were obtained and all lines confirmed as bona-fide SPT overexpressors exhibited the described seedling and dormancy phenotypes.

RNA Extractions and Real-time RT-PCR. Unless otherwise stated chemicals were purchased from Sigma (Poole, UK). RNA was isolated from dry, imbibed and germinating seeds using a protocol based on a borate extraction [31]. Briefly, 150 mg of seed (based on dry seed weight) was ground and extracted with 1 ml of frozen XT buffer (0.2M sodium borate, 30 mM EGTA, 1% SDS, 1% sodium deoxycholate, 2% polyvinylpyrollidone, 10 mM DTT, 1% IGEPAL pH 9.0) in a pestle and mortar. This was allowed to thaw and treated with 40 μl proteinase K (PCR grade, Roche, UK) for 90 mins at 42° C. followed by precipitation on ice for 1 hour with 80 μl 2M potassium chloride. The supernatant was collected after centrifugation at 4° C. The RNA was precipitated from the supernatant at −20° C. for 2 hours with 360 μl 8M lithium chloride. The RNA was collected by centrifugation at 4° C. and redissolved in 100 μl water. The RNA was further purified using the clean-up protocol of the RNeasy Plant RNA isolation kit (Qiagen), following the manufacturer's protocol. First strand cDNA was synthesised using 5 μg of total RNA in 20 μl reactions, Superscript II Reverse Transcriptase (Invitrogen) and random primers following manufacturer's instructions, and 180 μl water added before the PCR step.

Real-Time RT-PCR was performed using SYBR-green as described [32] using 2 μl of the diluted cDNA template and the following primers for the SPT, GA3ox1, and GA3ox2 cDNAs: SPTF: 5′-ccttacttcacccgtggagatg-3′ SPTR: 5′-gcgttggaatgaccaatgttc-3′ GA3OX1F: 5′-aagtggacccctaaagacgatct-3′ GA3OX1R: 5′-gtcgatgagagggatgttttcac-3′ GA3OX2F: 5′-tgagttcctcaccggaagtctt-3′ GA3OX2R: 5′-cgagccgccttgagctt-3′. All data points represent the mean and standard deviation of three independent determinations.

### Plasmid Construction and Generation of SPT-12xHA Overexpressing Lines

To construct the HA-tagged SPT, the cDNA of SPT (Accession no. AF319540) was PCR-amplified using pfu-Turbo™ DNA polymerase (Stratagene, La Jolla, US). Primers were 5′-gcgacgcgtaattactactaccatgatatcacagagagaagaa-3′and 5′-gcggggcccagtaattcgatcttttaggt-3′ respectively, introducing a MluI and an ApaI restriction site (bold). The PCR product was sequenced, cut in the introduced restriction sites and ligated into the binary plasmid pGT35SHA (R. Kannangara and I. A. Graham, unpublished), containing a double 35S enhancer and in frame with a 12xHA epitope-tag. This was introduced into Agrobacterium strain GV3101::pMP90, which was used to transform plants of A. thaliana ecotype Landsberg erecta using the floral-dip. Transgenic plants were selected for basta resistance on soil by spraying with KASPAR (Certis, Sutton on Derwent, UK). Three independent lines were produced, all of which displayed the long hypocotyl phenotype described for 35S:SPT.

### Protein Extractions and Western Blotting

T2-seedlings were grown in continuous white light (50 μM m⁻² s⁻¹) at 20° C. for 5 days on filter paper placed on ½ MS plates. 1 ml of 100 μM of cycloheximide (Sigma, Poole, UK) was added to the surface of the filter papers and plates were either placed in continuous white light or darkness at 20° C. Seedlings were harvested at 0, 3, 6 and 9 hours after treatment. Total protein was extracted by grinding ˜100 seedlings in a mortar and pestle under liquid nitrogen, adding 200 μl extraction buffer (100 mM Tris-HCl, pH 8, 50 mM EDTA, 50 mM NaCl, 0.7% (w/v) SDS, 1 mM DTT, 1 mM PMSF and protease inhibitor cocktail (Sigma, St. Louis, USA), heating for 10 min at 65° C. and clarifying by centrifugation at full speed for 10 min in a microfuge. Protein extracts were separated by SDS-PAGE (10%) and transferred to nitrocellulose membrane (Bio-Rad, Hercules, Calif.). A rat anti-HA monoclonal antibody 3F10 (Roche, Penzberg, Germany) was applied in a dilution of 1:5000. The immunoreactive polypeptides were visualized with an alkaline-phosphatase conjugated goat anti-rat antibody (abcam, Cambridge, UK). Signal intensity was quantified using adobe photoshop.

### EXAMPLE 1

### SPATULA Controls the Germination Response to Cold and Light

In order to isolate factors involved in seed germination control we isolated mutants in uncharacterised regulatory factors represented in seed EST collections and microarray data. One of these was the bHLH transcription factor SPATULA (SPT), previously characterised for its role in fruit development [Alvarez and Smyth, 1999; Heisler et al., 2001]. spt mutants are known to exhibit short siliques with a reduced pollen transmitting tract, yet the expression of SPT in the seed during germination, plus vegetative and non-fruit reproductive tissues indicated a wider role for SPT than previously described. In the seed SPT expression is induced during imbibition and continues to rise until germination has been completed (FIG. 1a). In order to further investigate the role of SPT in the seed, we obtained mutants of the SPT locus and analysed the effects of SPT disruption on seed dormancy and germination. A first allele of SPT, designated spt-10, was obtained from the Cold Spring Harbour collection. This contains a stable transposon insertion after the fourth predicted codon in the first exon, and the full length transcript could not be detected indicating that this likely represents a null allele. The siliques of spt-10 closely resemble those of the previously described spt loss-of-function mutants spt-1 and spt-3. The second, spt-2 has been previously characterised and is predicted to result in an amino acid substitution in the putative DNA binding domain of SPT (FIG. 1B). Interestingly, spt-2 mutants exhibit a stronger fruit phenotype than putative spt null alleles, suggesting that spt-2 has a dominant-negative effect on fruit development [13].

Using spt-10 and spt-2 we analysed the role of SPT in the control of seed dormancy and germination. Freshly harvested wild type (Ler) seed exhibited dormancy, and did not germinate without both light and cold stratification (FIG. 1C). Both treatments were always found to be necessary to induce germination in dormant seed. Although freshly harvested spt-10 mutant seeds were mostly dormant in the dark, they displayed a consistent strong reduced dormancy phenotype in the light: i.e. in the presence of light spt-10 seed did not require cold stratification for germination (FIG. 1C). In contrast, the germination of freshly harvested spt-2 seeds resembled wild type in both the light and the dark. After cold stratification both wild type and the spt-10 mutant germinated at high frequency in the light, while spt-10 also germinated at low frequency in the dark. Surprisingly, freshly harvested spt-2 seeds were found to be completely unresponsive to stratification, both in the presence of light or if maintained in the dark. Seeds heterozygous for spt-2 showed a response to stratification that was intermediate between spt-2 and wild type (FIG. 1D), hence we concluded that in the context of seed germination spt-2 behaves as a semi-dominant gain-of-function mutant.

Next we examined the germination of afterripened wild type, spt-2 and spt-10 seed. After 6 months storage Ler still required light for germination, but no longer cold stratification (FIG. 1C). Loss of SPT appeared to affect the light dependency of afterripened seed, as unlike wild type afterripened spt-10 mutant seed germinated at a significant rate in the dark. Strikingly, spt-2 seeds stored for a similar period now behaved like wild type, germinating at high frequency in the light, but not the dark. Hence we concluded that the primary consequence of the spt-2 mutation is the attenuation of the stratification response, as spt-2 only negatively affects germination while stratification is essential. Indeed experiments on partially afterripened spt-2 seed confirmed that no promotive effect of stratification is ever observed on the germination of the spt-2 mutant seed (data not shown). The spt mutant phenotypes were not maternally inherited ruling out a role for SPT in the seed coat control of dormancy [Debeaujon et al., 2000]. These data show that SPT has an important function in the control of seed germination in response to cold stratification, and as such is the first regulatory gene to be described with such a role.

To further enhance our understanding of the role of SPT in the control of dormancy breakage, the germination of freshly harvested wild type and spt-10 mutants was analysed over a range of stratification times in the dark, or after 10 seconds of red light (FIG. 2A). In agreement with previous results little wild type germination was seen in the dark, with or without stratification, and 3 days stratification only promoted a modest increase in spt-10 germination in the absence of light. A combination of three days stratification and red light was required for significant germination of Ler. In the spt-10 mutant red light promoted significant germination without any cold stratification at all, and short periods of chilling promoted high levels of germination. Hence we concluded that the primary consequence of loss of spt-10 function was a lack of chilling requirement for germination, and hypersensitivity to applied chilling. This was manifested predominantly in the elevation of the spt-10 germination response to light (FIG. 2A). Such a phenotype is consistent with the established role for stratification in potentiating the response of wild type seeds to light. To examine if the enhanced response of SPT to light was due to light stable phytochrome control, the far-red reversibility of red light induced germination was investigated in the spt-10 mutant after 3 days stratification (FIG. 2B). In this experiment red light induced high levels of germination in wild type and spt-10, but not the phyB-1 mutant which is defective in red light signalling. Red light induced germination was fully reversible by a far red pulse in wild type, phyB-1 and spt-10, confirming that red light induced germination in spt-10 is dependent on light stable phytochrome action. However, spt-10 seeds also exhibit a low rate of germination in the dark, which is unaffected by a far red pulse, demonstrating that one function of SPT is to repress germination to a small but significant extent in the absence of light. Thus SPT has a role in coupling seed germination to the light response.

### EXAMPLE 2

### SPATULA is a Repressor of GA3ox Expression in Dormant Seeds

One of the key targets of light and cold signalling in the seed is the promotion of GA biosynthesis through the transcriptional regulation of GA3ox [Yamaguchi et al., 1998; Yamauchi et al., 2004]. To investigate the possibility that SPT functions in the light and temperature control of GA3ox expression we used real-time RT-PCR to determine the expression of both GA3ox1 and GA3ox2 in imbibed seed of wild type and the spt mutants in the dark and light, 24 h after imbibition at 20° C., or 24 h after transfer to 20° C. following 3 nights stratification (FIG. 3). At this time point the expression of both GA3ox isoforms peaks in the imbibed seed [Yamaguchi et al., 1998]. In contrast to previous analyses we found that both GA3ox1 and GA3ox2 required the synergistic effect of light and stratification for high expression in dormant Ler seeds, and that neither treatment alone was sufficient to induce high expression of either isoform. This correlates well with observed germination under these conditions (FIG. 1C). In unstratified seeds maintained in the dark GA3ox transcript levels were low in all genotypes. In unstratified seeds exposed to light no increase in GA3ox expression was observed in the wild type, yet in the spt-10 mutant five and twenty fold increases were observed in GA3ox1 and GA3ox2, respectively when compared to Ler. This correlates well with the germination phenotype of the spt-10 mutant, and demonstrates that SPT functions as a repressor of both GA3ox1 and GA3ox2 expression in dormant seeds in the light. In agreement with observed germination, no increase was seen in the spt-2 mutant. spt-10 also had increased GA3ox transcript levels in dark stratified seeds, where the spt-10 mutant also exhibits slightly increased germination compared to wild type. When stratified seeds were exposed to light a large increase in GA3ox expression was seen in Ler and spt-10. Strikingly, after light and stratification the increase in GA3ox transcript levels is completely repressed in spt-2, in agreement with the failure of the spt-2 mutant to germinate under these conditions (FIG. 1C). These findings confirm that SPT is a key negative regulator of GA3ox expression in imbibed seeds. In general a strong correlation between germination frequency and GA3ox transcript levels was observed, with the minor exception that GA3ox1 appeared to have higher expression in dark stratified seeds of both wild type and the spt-10 mutant than would be expected from their respective germination frequencies. This may point to the existence of a second mechanism attenuating GA3ox1 function in the dark, or simply reflect the increased importance of the regulation of the sensitivity to GA under these conditions.

### EXAMPLE 3

### SPATULA Overexpression Disrupts the Light Response in Seeds and Seedlings

In order to complement the mutant analysis we investigated the effect of SPT overexpression on seed germination and plant growth. Our data demonstrate that 35S::SPT seedlings display a phenotype that is consistent with the proposed role of SPT in the response of seed germination to cold and light. 15 independent transgenic lines containing the full length SPT cDNA fused to a double Cauliflower Mosaic Virus (CaMV) 35S promoter displayed a clear long hypocotyl phenotype when grown in white light and the dark (FIG. 4A, B). The light-grown phenotype closely resembles that of phyB loss of function mutants [Reed et al., 1993]. In addition, when partially afterripened wild type and 35S:SPT seed were germinated in white light, a clear increase in seed dormancy was observed in SPT overexpressors compared to wild type lines treated identically (FIG. 4C). However, unlike spt-2, the germination of SPT overexpressing seeds was clearly restored by a combination of stratification and constant white light. To further investigate the effect of SPT overexpression on the light regulation of germination, wild type, 35S:SPT and phyB-1 seed were stratified and germinated in the dark, or after pulses of red light of increasing duration (FIG. 4D). Whilst a strong promotion of germination was observed in wild type after short pulses of red light, both 35S:SPT and phyB-1 showed a decreased response to red light. In fact 35S:SPT was less sensitive to light than phyB-1, suggesting that SPT overexpression affects phytochrome pathways in addition to phyB. When GA3ox expression was measured in dormant wild type and 35S:SPT lines 24 hours after imbibition in the light low expression was observed in unstratified seed of both genotypes (FIG. 4E). A strong increase was seen in wild type after stratification, in agreement with previous experiments. In 35S:SPT GA3ox levels also increased after stratification, but this increase was at least two-fold reduced compared to wild type. Hence we concluded that although SPT overexpression has a repressive effect on GA3ox expression in light stratified seed, the effect is not sufficiently strong to maintain GA3ox expression below the level required for germination.

### REFERENCES

Yamaguchi, S., Smith, M. W., Brown, R. G., Kamiya, Y., and Sun, T. (1998) Phytochrome regulation and differential expression of gibberellin 3beta-hydroxylase genes in germinating Arabidopsis seeds. Plant Cell 10, 2115-2126.

Yamauchi, Y., Ogawa, M., Kuwahara, A., Hanada, A., Kamiya, Y., and Yamaguchi, S. (2004) Activation of gibberellin biosynthesis and response pathways by low temperature during imbibition of Arabidopsis thaliana seeds. Plant Cell 16, 367-378.

Heisler, M. G., Atkinson, A., Bylstra, Y. H., Walsh, R., and Smyth, D. R. (2001) SPATULA, a gene that controls development of carpel margin tissues in Arabidopsis, encodes a bHLH protein. Development 128, 1089-1098.

Alvarez, J., and Smyth, D. R. (1999) CRABS CLAW and SPATULA, two Arabidopsis genes that control carpel development in parallel with AGAMOUS. Development 126, 2377-2386.

Debeaujon, I., Leon-Kloosterziel, K. M., and Koornneef, M. (2000) Influence of the testa on seed dormancy, germination, and longevity in Arabidopsis. Plant Physiol. 122, 403-414.

Reed, J. W., Nagpal, P., Poole, D. S., Furuya, M., and Chory J. (1993) Mutations in the gene for the red/far-red light receptor phytochrome B alter cell elongation and physiological responses throughout Arabidopsis development. Plant Cell 5, 147-157.

1. A plant comprising a genetically modified cell wherein the genome of said cell is modified by the inclusion of a nucleic acid molecule comprising a variant of SEQ ID NO: 1, wherein said nucleic acid molecule encodes a transcription factor polypeptide that is a variant polypeptide from that encoded by SEQ ID NO: 1, which variant polypeptide comprises an amino acid deletion or substitution of amino acid residue 209.

2. A plant comprising a genetically modified cell wherein the genome of said cell is modified by the inclusion of a nucleic acid molecule that hybridises under stringent hybridisation conditions to SEQ ID NO: 1 and which encodes a transcription factor polypeptide that is a variant polypeptide from that encoded by SEQ ID NO: 1, which variant polypeptide comprises an amino acid deletion or substitution of amino acid residue 209.

3. A plant according to claim 1 wherein said substitution is the replacement of amino acid residue 209 with a basic amino acid residue

4. A plant according to claim 1 wherein said amino acid residue 209 is arginine.

5. A plant according to claim 4 wherein said arginine amino acid residue is replaced with a basic amino acid residue that is not arginine.

6. A plant according to claim 5 wherein said basic amino acid residue is lysine or histidine.

7. A plant according to claim 1 wherein said plant comprises a nucleic acid molecule that encodes a transcription factor the activity of which is modulated.

8. A plant according to claim 1 wherein transcription factor activity is increased when compared to a non-transgenic reference plant of the same species.

9. A plant according to claim 1 wherein said nucleic acid molecule is a vector adapted for transformation of said plant cell.

10. A plant according to claim 9 wherein said nucleic acid molecule is controlled by a seed specific promoter.

11. A plant according to claim 1 wherein said plant has reduced germination when compared to a non transgenic reference plant of the same species.

12. A plant according to claim 1 wherein said plant has reduced response to cold stratification when compared to a non transgenic reference plant of the same species.

13. A seed obtained from a plant according to claim 1.

14. A plant cell wherein said cell is modified by the inclusion of a nucleic acid molecule comprising a variant of SEQ ID NO: 1, wherein said nucleic acid molecule encodes a transcription factor polypeptide that is a variant polypeptide from that encoded by SEQ ID NO: 1, which variant polypeptide comprises an amino acid deletion or substitution of amino acid residue 209.

15. A plant cell wherein said cell is modified by the inclusion of a nucleic acid molecule that hybridises under stringent hybridisation conditions to SEQ ID NO: 1 and which encodes a transcription factor polypeptide that is a variant polypeptide from that encoded by SEQ ID NO: 1, which variant polypeptide comprises an amino acid deletion or substitution of amino acid residue 209.

16. A method for the identification of a locus associated with sprouting/precocious germination wherein said locus is associated with a nucleic acid sequence selected from the group consisting of:
a) a nucleic acid molecule comprising a nucleic acid sequence as represented in SEQ ID NO: 1;
b) a nucleic acid molecule that hybridises to the nucleic acid molecule in a) under stringent hybridisation conditions and that encodes a polypeptide with transcription factor activity; and
c) a nucleic acid molecule comprising a nucleic acid sequence that is degenerate as a result of the genetic code to the sequences as defined in (a) and (b) above;
the method comprising the steps of:
i) providing a sample comprising a plant cell wherein said plant cell is derived from a plant that does not express a sprouting/precocious germination phenotype; and
ii) comparing the sequence of the nucleic acid molecule in said sample to a nucleic acid sequence of a nucleic acid molecule of a plant that does express a sprouting/precocious germination phenotype.

17. A method to produce a plant variety that does not express a sprouting/precocious germination phenotype comprising the steps of:
i) mutagenesis of wild-type seed from a plant that does express a sprouting/precocious germination phenotype;
ii) cultivation of the seed in i) to produce a first generation and subsequent generations of plants;
iii) obtaining seed from the first generation plant or subsequent generations of plants;
iv) determining if the seed from said first generation plant or subsequent generations of plants does not express a sprouting/precocious germination phenotype;
v) obtaining a sample and analysing the nucleic acid sequence of a nucleic acid molecule selected from the group consisting of:
a) a nucleic acid molecule comprising SEQ ID NO: 1;
b) a nucleic acid molecule that hybridises to the nucleic acid molecule in a) under stringent hybridisation conditions and that encodes a polypeptide with transcription factor activity; and
c) a nucleic acid molecule comprising a nucleic acid sequence that is degenerate as a result of the genetic code to the sequences as defined in (a) and (b) above; and
vi) comparing the sequence of the nucleic acid molecule in said sample to a nucleic acid sequence of a nucleic acid molecule of a plant that does express a sprouting/precocious germination phenotype.

18. A method according to claim 17 wherein said nucleic acid molecule is analysed by a method comprising the steps of:
i) extracting nucleic acid from said mutated plants;
ii) amplification of a part of said nucleic acid molecule by a polymerase chain reaction;
iii) forming a preparation comprising the amplified nucleic acid and nucleic acid extracted from wild-type seed to form heteroduplex nucleic acid;
iv) incubating said preparation with a single stranded nuclease that cuts at a region of heteroduplex nucleic acid to identify the mismatch in said heteroduplex; and
v) determining the site of the mismatch in said nucleic acid heteroduplex.

19. A method according to claim 16 wherein said nucleic acid molecule comprises SEQ ID NO: 1.

20. A method according to claim 19 wherein said nucleic acid molecule consists of SEQ ID NO: 1.

21. A method according to any claim 16 wherein said plant cell or seed is from wheat, barley or oil seed rape.

22. The use of a gene comprising SEQ ID NO: 1, or a nucleic acid molecule that hybridises to SEQ ID NO: 1 and encodes a polypeptide with transcription factor activity, as a quantitative trait locus.
